(19) United States
(12) Patent Application Publication
(10) Pub. No.: US 2014/0212943 A1
(43) Pub. Date: Jul. 31, 2014

(54) METHOD FOR IN-VITRO TREATMENT OF DIFFERENTIATED OR UNDIFFERENTIATED CELLS BY APPLICATION ELECTROMAGNETIC FIELDS

(71) Applicants: Salvatore Rinaldi, Scandicci (IT); Vania Fontani, Scandicci (IT)

(72) Inventors: Salvatore Rinaldi, Scandicci (IT); Vania Fontani, Scandicci (IT)

(21) Appl. No.: 14/238,499

(22) PCT Filed: Aug. 10, 2012
(86) PCT No.: PCT/IB2012/054078

§ 371 Date: Feb. 12, 2014

(30) Foreign Application Priority Data
Aug. 12, 2011 (IT) FI2011A000179

(51) Int. Cl. C12N 13/00

(52) U.S. Cl. CPC C12N 13/00
USPC 435/173.1

(57) ABSTRACT

A process is described for the treatment of stem cells or differentiated cells by application of radiofrequency electro-magnetic fields, by means of an electromagnetic field generator (10), comprising a power supply (11), at least one antenna (12) adapted to radiate a scattered electromagnetic field (13) of a power less than 100 mW, a modulator (14) associated with said generator (10) and adapted to modulate the emission thereof, and at least one convector electrode (15), adapted to be direct the radiofrequency currents induced by said electromagnetic field (13), and applied in proximity of said stem or differentiated cells.

### FIELD OF THE INVENTION

The present invention relates to the field of in-vitro cell treatment through the application of electromagnetic fields.

### PRIOR ART

As is known, electromagnetic fields of various frequencies are widely used in the field of telecommunications, but in addition to this principal use, the aforementioned radiofrequencies have also recently proved capable of interfering with processes of cellular homoeostasis.

In particular C. Ventura et al., in Turning on stem cell cardiogenesis with extremely low frequency magnetic fields (FASEB J. 19(1):155-157 (2005), have demonstrated that mouse embryonal stem (ES) cells exposed to extremely low frequency electromagnetic fields (50 Hz, 0.8 mTrms) considerably increased the transcription of cardiogenic and cardiospecific genes by raising the production of stem cell-derived cardiomyocytes.

The reprogramming of both mouse and human adult somatic non-stem cells , such as fibroblasts, into induced pluripotent cells (iPS, “induced Pluripotent Stem Cells”) and subsequent differentiation of these reprogrammed cells into specialised mature cells, has opened the way to new prospects in the field of regenerative medicine (Takahashi, K.; Tanabe, K.; Ohnuki, M.; Narita, M.; Ichisaka, T.; Tomoda, K.; Yamanaka, S. Induction of pluripotent stem cells from adult human fibroblasts by defined factors. Cell 131(5):861-872; 2007). More recently it has been demonstrated that mouse fibroblasts can be directly reprogrammed into a myocardiac phenotype without first passing through an iPS stage (Ieda, M.; Fu, J. D.; Delgado-Olguin, P.; Vedantham, V.; Hayashi, Y.; Bruneau, B. G.; Srivastava, D. Direct reprogramming of fibroblasts into functional cardiomyocytes by defined factors. Cell 142(3):375-386; 2010). Such reprogramming experiments, both via an intermediate iPS stage and via direct phenotypic transformation, are always performed by means methods of transferring a specific gene set via viral vectors.

These approaches are therefore hampered both by the complexity of the method and, above all, by the potential risks associated with the use of viral vectors. Moreover, with both reprogramming with intermediate iPS and direct reprogramming the differentiation yield obtained is extremely low, usually below 1%. This low differentiation yield is furthermore associated with the high tumorigenic risk of the reprogrammed cell population remaining at a quasi-embryonic undifferentiated stage.

It would obviously be of great interest to be able to treat the cells by potentiating the totipotentialities thereof, or actually by bringing differentiated cells to their condition of totipotency without having to apply chemical or genetic stimuli cell, so as to interfere as little as possible with the cell structure, which would enable, among other things, all the ethical/legal problems associated with the use of stem cells to be overcome.

### SUMMARY OF THE INVENTION

A process is described for the treatment of differentiated or undifferentiated cells through the application of electromagnetic fields at low-power radiofrequency.

### BRIEF DESCRIPTION OF THE FIGURES

FIG. 1 illustrates diagrammatically an apparatus used in the process according to the invention.

FIG. 2 illustrates the effects of stimulation with electromagnetic fields at low-power radiofrequency on the expression of genes which orient a stem cell towards cardiogenic, skeletal myogenic and neurogenic developmental lines.

FIG. 3 illustrates how stimulation with electromagnetic fields at low-power radio-frequencies lowers the expression of marker genes of undifferentiated stem cells.

FIG. 4 illustrates how the exposure of cells to electromagnetic fields at low-power radiofrequencies modulates the expression of tissue-specific and stem-cell related proteins.

FIG. 5 shows the increase in spontaneously beating colonies obtained in the absence or presence of treatment according to the invention.

### DETAILED DESCRIPTION OF THE INVENTION

The present invention enables a response to the above-mentioned demands by virtue of a process of cellular differentiation using the action of low-power radio-frequency electromagnetic fields.

In particular the device which is the subject of European patent EP 1 301 241 may be used for generating the magnetic fields according to the invention.

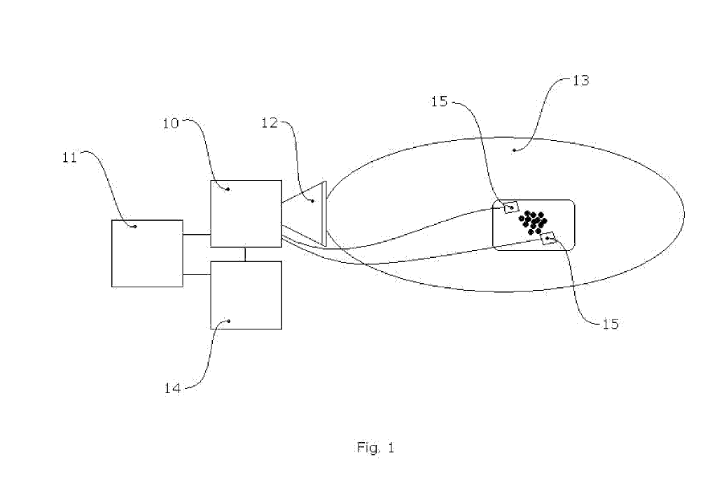

In brief, said device, which is diagrammatically represented in the attached FIG. 1, comprises a radiofrequency electromagnetic field generator 10 connected to a suitable power supply 11, at least one antenna 12 associated with said generator 10 and adapted to radiate a scattered electromagnetic field 13, a modulator 14 associated with said generator 10 and adapted to modulate the emission thereof, and at least one convector electrode 15, associated with said generator 10, possibly through the modulator 14, and adapted to direct the radiofrequency currents induced by the above-mentioned electromagnetic field 13.

The present invention provides for irradiation of the cellular material to be treated by using the device which is the subject of European patent EP 1 301 241, or a similar device, in the vicinity of said cellular material, applying said convector electrode 15 in the proximity of said stem or differentiated cells, as shown diagrammatically in the attached FIG. 1.

Moreover, according to the invention “low power radiofrequency electromagnetic fields” is used to mean radiofrequency electromagnetic fields characterised by a radiated power measured at the low-entity emitter, for example less than 100 mW, preferably less than 50 mW, more preferably less than 10 mW. In particular, it is noted that, according to the invention, stem cells subjected to the action of radiofrequency electromagnetic fields as described above, in a neutral growth medium, are capable of increasing their total pluripotency and, once cultivated on a suitable medium, not simply of developing into cells of any tissue type (muscle, bone, nerve, gland, etc). This aspect is especially evident if the cardiogenic effect produced by the magnetic fields described above are considered. Indeed, the action of the magnetic field is capable of inducing not only the appearance of cardiac myocytes having spontaneous contractile activity, but also the aggregation of said myocardial cells to form true myocardial tissue wherein the overall contractile activity is organised in a helical course, starting from a pace-setting trigger focus.

It is also noted that by applying the method according to the invention to differentiated cells, for example fibroblasts, in a suitable culture medium, the cells thus treated reverted to behaving like totipotent stem cells.

Hereinafter we report examples wherein stem cells treated according to the methods and with the device described above, developed totally into the respective nerve, skeletal muscle and myocardial cells, and wherein multipotent adult human stem cells were rendered pluripotent.

Examples of fibroblast and oocyte therapy according to the invention are also reported.

### EXAMPLE 1

The device described in patent EP 1 301 241, which had been placed in a CO₂ incubator, was regulated in such a way as to emit electromagnetic radiation at a frequency approximately equal to 2.4 GHz, and the convector electrodes were immersed in the culture medium wherein R1 mouse ES cells were already present.

The distance between the 2.4 Ghz frequency source and the culture medium was approximately 35 cm. The amount of electromagnetic radiation supplied was measured using a Tektronix 2754p spectrum analyser, by orientating the antenna thereof receiving most of the signal.

Taking into account a duration for each individual radiofrequency emission of 200 ms and a switch-off interval of 2.5 s, the following results were obtained: the emitted power P is approximately 2 mW, the electric field E is equal to 0.4 V/m, the magnetic field M is approximately 1 mA/m, and the specific absorption rate, or SAR, approximately 0.128 μW/g. Having ascertained α=1 A/Vm , and p=1000 Kg/m³, the electromagnetic current density within the culture medium during the irradiation by the device described above is equal to J=30 μA/cm². Notwithstanding that the electromagnetic field measured around the device is highly irregular due to the presence of the metallic walls of the incubator, it was possible to measure the maximum intensity within the incubator. Radiated power values equal to 400 μW/m² were measured at a distance of approximately 35 cm from the emitter and within a very restricted area around the receiving antenna of the meter.

The R1 ES cells were maintained in an undifferentiated state by cultivating them on a layer of mouse embryo fibroblasts that had been mitotically inactivated in the presence of Knockout DMEM containing 15% FBS in a final concentration of 100 U/ml LIF.

Otherwise, cellular differentiation was conducted in accordance with the usual methods, by placing the cells on plates (Costar ultra low attachment clusters) containing the culture medium in the absence of LIF, after two days of culture the resultant embryoid bodies (Ebs) were placed on tissue culture plates.

### Gene Expression

The total RNA was isolated using triazole reagent in accordance with the guidelines of the manufacturer (Invitrogen). The total RNA was dissolved in water devoid of RNA-ase, and, to perform RT-PCR, cDNA was synthesised in 50 μl with 1 μl total RNA and MuMLV inverse transcriptase (RT) as instructed by the manufacturer (Invitrogen).

Quantitative PCR in real time was performed using an iCycler Therma Cycler (Bio-Rad). 2 μl cDNA was amplified into 50 μl using Platinum Supermix UDG (Invitrogen), 200 nM of each primer, 10 nM of fluorescein (bio-rad) and Sybr Green. Following an initial denaturation phase at 94° C. for 10 minutes, thermal cycling was initiated. Each cycle consisted of 15 sec at 94° C., 30 sec at 55-59° C., 30 sec at 60° C., and the fluorescence was read off at the end of this phase. In the described example, all the primers used were of the Invitrogen type, but analogous results are achieved using different primers.

To evaluate the quality of the PCT samples in real time, analysis of the fusion curve was conducted after each sample.

The respective expression was determined using the “delta-CT” method with GAPDH as reported in Maioli M. et al. “Hyaluronam esters drive Smad gene expression and signalling enhancing cardiogenesis in mouse embryonic and human mesenchymal stem cells PloS One 5(11):e15151 (2010).

### Immunoblotting Analysis

ES cells were harvested and pelleted in PBS, then the pellets were lysed with buffer for extraction from the cells (Invitrogen). The cellular lysate was subjected to electrophoresis on 10% Novex Tris-glycine polyacrylamide gel (Invitrogen, CA) in MOPS SDS buffer, using an XCeII Sure Lock© Mini-Cell in accordance with the manufacturer's instructions.

Following transfer of the proteins onto membranes in polyvinylidene difluoride (PVDF) (Invitrogen, CA), saturation of the membranes and washing, the immune reactions were conducted for 1 hour at ambient temperature in the presence of primary antibodies (anti-GATA4 antisera, Myo, β-3-tubulin, sox2 and NANOG), diluted 1:1000. Following further washing, the membranes were incubated with a secondary antibody (abCAM) anti-rabbit (Sox2 and NANOG) or anti-mouse (GAT4, MyoD, β-3-tubulin) conjugated with horseradish peroxidase (HRP). The expression of labelled proteins was measured with a detection system for chemoluminescence (using ECL Western blotting the agents from Amersham Biosciences).

### Immunostaining

R1 cells cultivated for 3 days with or without application of the radiofrequency electromagnetic fields were treated with trypsin, and the resultant suspension was cultured at low density to enable visualisation of the individual cells. The cultures were fixed with 4% paraformaldehyde. The cells were exposed for 1 h at 37° C. to mouse anti-actinic, α-sarcomeric monoclonal antibodies, β-3-tubulin, MyoD or Myogenin or rabbit polyclonal antibodies to the heavy chain of myosin, and stained at 37° C. for 1 h with goat IgG conjugated with fluorescein.

The microscopic verification was conducted using a Leica confocal microscope (LEICA TCSSP5), and the DNA was visualised using propidium iodide (1 μg/ml).

### Analytical Data

The statistical analysis of the data was conducted using Student's t-test, taking a value of p<0.05 as the limit of significance.

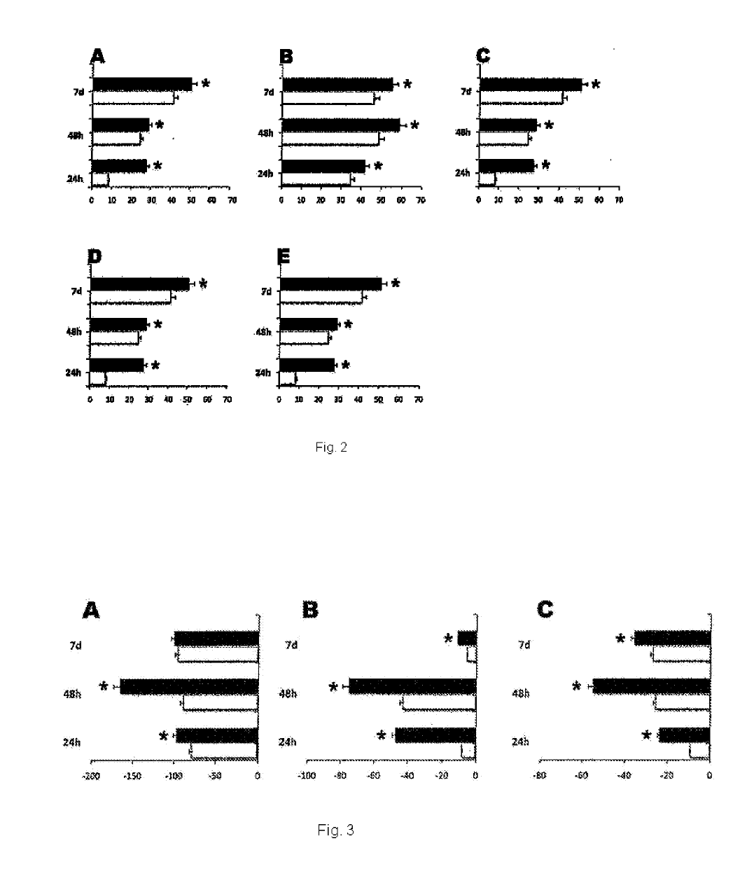

The R-PCR in real time demonstrated a significant increase in the expression of the prodynorphin gene after 24 hours exposure to radiofrequency electromagnetic fields, an effect that was still evident after 2 days (see FIG. 2A) (the asterisk refers the measured values for the treated cells).

Surprisingly, in the case of protracted electromagnetic stimulation over 48 h, the effect extended over the next 7 days (see FIG. 2A) and is compatible to that obtained with continuous exposure for 10 days (not shown in the drawing). The effect of electromagnetic stimulation on prodynorphin transcription is of considerable interest, considering the capacity of this gene, and of its associated product (dinorfin B) to control the homoeostasis of cytosolic Ca 2+ and the contract to Italy in adult cardiomyocytes cells and for inducing the transcription of cardiogenic genes in ES cells via the activation of autocrine circuits and “intracrine” signaling by opioid receptors.

By underlining the central role of the gene prodynorphin in cardiogenesis, the electromagnetically stimulated ES cells showed a significant increase in the expression of GATA4 and Nkx-2.5 (FIG. 2B, C). These genes coding respectively for a zinc finger dominium and a homeodominium are essential for cardiogenesis in various animal species including humans.

The transcription of myoD and neurogenin 1 was also increased in a similar way, in respect of both times and persistence after stimulation (see FIG. 2D, E).

It is also known that to regulate proliferation and differentiation, the ES cells must be capable of closely controlling the transcription of numerous factors including Sox2, NANOG, and Oct4.

Sox 2 is able to act in synergy with Oct3/4 to activate Oct-Sox stimulators which regulate the expression of specific genes of pluripotent stem cells such as NANOG, Oct3/4 and Sox2 itself.

Suppression of the activity of the Oct4 gene in mouse embryos impedes proliferation of the internal cell mass (ICM) and promotes differentiation into trofectoderm. Once expressed, NANOG blocks differentiation.

Negative regulation of NANOG is therefore necessary to sustain differentiation during the development of ES cells.

Early stages of ES cells differentiation, following removal of LIF, imply a sub-regulation of the expression of Sox2 (see FIG. 3).

Although with different times, similar effects similar effects are encountered on the expression of the genes Oct4 and NANOG following exposure to electromagnetic waves (see FIG. 3B, C).

To evaluate whether the transcriptional responses observed indicate an increase in differentiation, the effects of the magnetic fields were verified on the expression of tissue-specific protein markers.

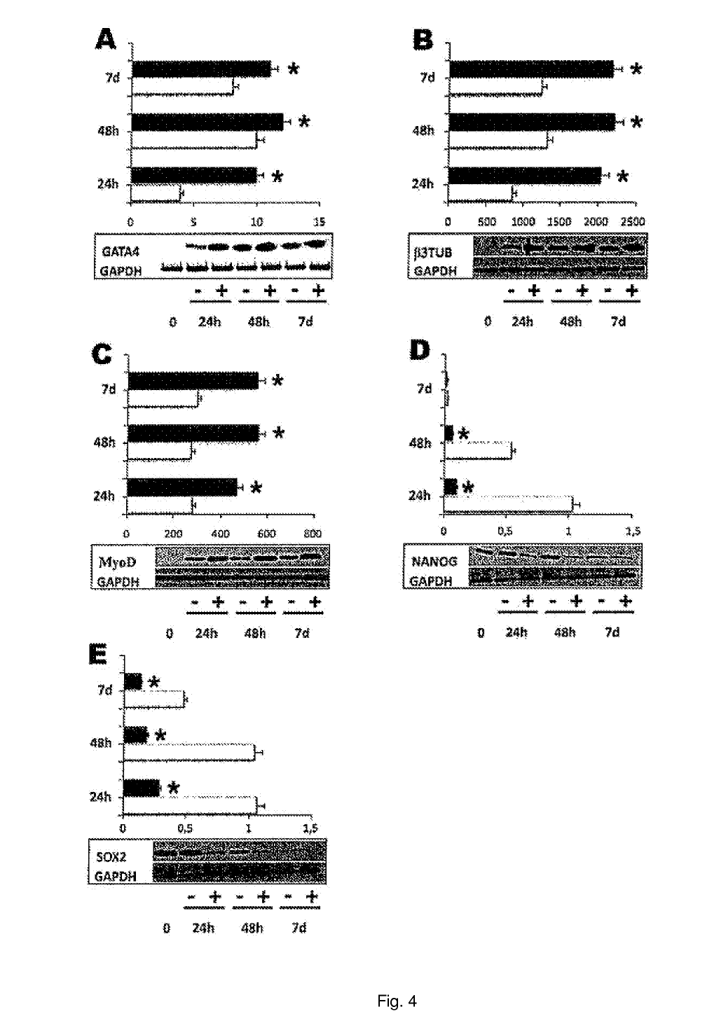

The Western blot analysis has revealed that GATA4, β-3-tubulin and myoD, indicators respectively of cardiac, neuronal and skeletal-muscle differentiation, are significantly overexpressed in cells treated with electromagnetic stimulation, as compared with non-stimulated cells (FIG. 4A-C). As for the transcriptional effects, said increase was still evident 2 days after the treatment, and persisted for the next 7 days even in the absence of stimulation (FIG. 4A-C).

In cells exposed to radiofrequency electromagnetic fields, the expression of Sox2 and NANOG reflected the increased transcriptional responses following stimulation with radiofrequency electromagnetic fields in being significantly sub-regulated in exposed cells as compared with non-exposed cells (FIG. 4D, E).

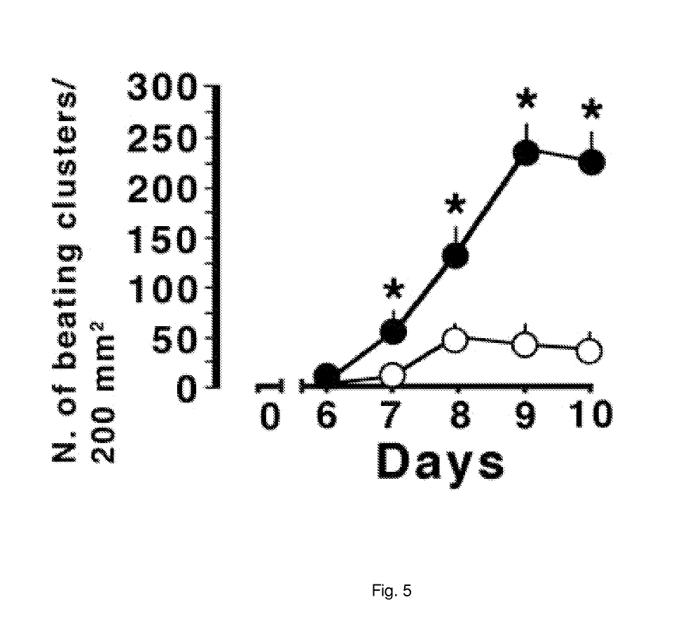

The formation of a cardiac phenotype was furthermore deduced by observing that stimulation with radiofrequency electromagnetic fields resulted (see FIG. 5) in a considerable increase in the number of false dating colonies. spontaneously deriving from aggregated cells such as EBs for 48 hours following removal of LIF, in the absence (white circles) or in the presence (black circles), of treatment according to the invention.

All the data collected therefore prove that, following exposure of ES cells to radiofrequency electromagnetic fields, differentiation occurs.

Differently from the electromagnetic fields used in the literature, the treatment with radiofrequency electromagnetic fields according to the invention, in the modalities described in the present invention, allowed the consistent increase in the differentiation pluripotentiality which, in the example given, involves three lines of development: cardiogenesis, neurogenesis and skeletal-muscle myogenesis without the intervention of chemical or biological agonists, or of genetic engineering; obviously the differentiation can involve other lines of cell development by acting according to the known techniques provided that the method according to the invention is applied.

Furthermore, as already mentioned above, by repeating the experiment described in the example illustrated above under the same conditions but using differentiated cells such as, for example, fibroblasts instead of stem cells, the effect observed was that the treated cells were reprogrammed, reverting to behave like totipotent stem cells.

### EXAMPLE 2

Multipotent human-adult mesenchymal stem cells (having a degree of differentiation potentiality less than that of the embryonic stem cells considered for definitions of pluri-or totipotentiality), isolated from adipose tissue and from other sources such as bone medulla, dental pulp and foetal membranes of full-term placenta, when exposed to the described magnetic field according to the invention for 72 h and then cultivated in the absence of further exposures for 4 or 7 days (corresponding to 7 or 10 days from starting time 0), behaved like quasi-embryonic, pluripotent cells in that they acquired the capacity to differentiate, on equal terms with mouse embryo cells exposed to the magnetic field of the present invention, both in myocardial cells, neuronal cells and skeletal-muscle cells. These results indicate that the treatment conducted as stated in the present invention is able to convert human stem elements from a state of multipotency to an extremely more “plastic” state of pluripotency, therefore maximising the hypothetical prospects of cell therapy and regenerative medicine with adult human stem cells.

### EXAMPLE 3

Human fibroblasts were exposed to a magnetic field for 72 h. and then cultured in the absence of exposure for a further 4 or 7 days (corresponding to 7 or 10 days from starting time 0) stock the duration of each individual radiofrequency emission was 200 ms with a switching-off interval of 2.5 s. Under these experimental conditions, the percentage of cells which expressed beta-3-tubulin, a marker of neuronal differentiation, was above 16%, while the percentage of cells expressing myoD, a marker of skeletal-muscle differentiation, was more than 20%, and the percentage of cells expressing alpha-sarcomeric actinin, a marker of terminal myocardial differentiation, was actually over 30%.

Application of the process according to the invention to human skin fibroblasts in culture was able to:
(i) induce at the transcriptional level, the activation of tissue-specific genes such as Mef2c, Tbx5, GATA4, Nkx2.5 and prodynorphin, associated with cardiogenic orientation,
(ii) induce at the transcriptional level, the expression of MyoD, a key gene in skeletal myogenesis,
   (iii) induce at transcriptional level, the expression of neurogenin1, the orchestrator gene of neurogenesis. Treatment with the magnetic field, according to the present invention has furthermore induced a by facing effect on the transcriptional genes responsible for the condition of being a stem cell, and pluripotency.

In particular, within the course of the first 6-20 h, the treatment induced the expression of Oct4, Sox2, cMyc, NANOG, and Klf4, albeit causing transcriptional inhibition of the same genes after 24 h. This aspect is of considerable interest in that the magnetic-field treatment according to the invention has not “frozen” the human fibroblasts at an IPS stage, with persistent overexpression of stem-state genes which would have subsequently “braked” differentiation into mature cells. On the contrary, transcriptional inhibition, which is effected 24 h after the treatment described in the present invention on the above-mentioned genes, has enabled especially elevated yields of differentiation in the myocardiac, skeletal-muscle and neuronal direction to be obtained.

### EXAMPLE 4

The experiment was repeated as described above and with the use of oocytes, with the following results:
optimisation of the maturation of the oocytes, as demonstrated in the oocytes of horses, sheep and humans, with consequent improvement in nuclear and cytoplasmatic maturation and expansion of the cumulus cells, even by intracytoplasmatic injection of the spermatozoon (ICSI);
Improved vitality of atresic and/or degenerate oocytes;
Stimulation and improvement of cleavage, with an embryonal development at the fibroblast stage;
Embryonal stimulation pre- and post-freezing to facilitate subsequent fertilisation, even by intracytoplasmatic injection of the spermatozoon;
Treatment of embryos obtained by even by intracytoplasmatic injection of the spermatozoon, for cloning purposes;
Cell-line stimulation, to induce cloning and genetic modification;
Sperm stimulation for methods of in-vitro fertilisation
Stimulation even of sessile spermatozoa by methods of in-vitro fertilisation.

1-8. (canceled)

9. A process for treating differentiated cells to turn them into undifferentiated cells or for treating undifferentiated cells to turn them into differentiated cells, comprising the steps of:
providing an electromagnetic field generator comprising a power supply, at least one antenna capable to radiate a scattered electromagnetic field having a power lower than 100 mW measured at said antenna, a modulator associated to said generator and capable to modulate the emission thereof, at least one convector electrode capable to direct the radiofrequency currents induced by the above said electromagnetic field;
providing differentiated or undifferentiated cells to be treated;
placing said at least one convector electrode in close proximity of said differentiated or undifferentiated cells under treatment;
radiating said differentiated or undifferentiated cells under treatment by means of said electromagnetic field generator.

10. The process according to claim 9, wherein said radiofrequency electromagnetic fields have a power lower than 50 mW.

11. The process according to claim 10, wherein said radiofrequency electromagnetic fields have a power lower than 10 mW.

12. The process according to claim 9, wherein stem cells are used in a neutral growth medium.

13. The process according to claim 9, wherein differentiated cells are used.

14. The process according to claim 9 wherein:
R1 mice embryonic stem cells are placed in culture medium;
convector electrodes of a device according to claim 1 are immersed in the culture medium;
the cells are exposed to electromagnetic fields of 2 mW power;
the embryoid bodies developed after two days of culture are placed on tissue culture plates.

15. The process according to claim 12 wherein normal differentiated cells are used instead of stem cells.

16. The process according to claim 9 wherein ovocytes are used instead of stem cells.

17. The process according to claim 13 wherein said differentiated cells are fibroblasts.

18. The process according to claim 14 wherein normal differentiated cells are used instead of stem cells.

19. The process according to claim 13 wherein ovocytes are used instead of stem cells.

20. The process according to claim 14 wherein ovocytes are used instead of stem cells.
